(19) United States
(12) Patent Application Publication
(10) Pub. No.: US 2007/0270863 A1
(43) Pub. Date: Nov. 22, 2007

(54) Devices and methods for contouring an intervertebral space between vertebral members

(75) Inventor: Anthony J. Melkent, Memphis, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/407,494

(22) Filed: Apr. 20, 2006

(51) Int. Cl. A61B 17/00

(52) U.S. Cl. 606/79

(57) ABSTRACT

The present application is directed to devices and methods to shape an intervertebral space between vertebral members. In one embodiment, the device includes a guide with an elongated shaft and an enlarged distal section. An outer member may include first and second arms that are spaced a distance apart. Teeth may be positioned on outer surfaces of one or both of the first and second arms. The outer member may be movably positioned along the guide between first and second positions. The first position may include the teeth positioned over the shaft with the first and second arms being positioned a first distance apart. The second position may include the teeth positioned over the distal section with the first and second arms being positioned an expanded second distance apart.

### BACKGROUND

The present application is directed to devices and methods for contouring vertebral members, and more particularly, to devices and methods for contouring the shape of an intervertebral space between vertebral members.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various conditions may lead to damage of the intervertebral discs. The damage may result from a variety of causes including a specific event such as trauma, a degenerative condition, a tumor, or infection. Damage to the intervertebral discs and vertebral members can lead to pain, neurological deficit, and/or loss of motion.

Various procedures include replacing the intervertebral discs. These procedures often require contouring the endplates of the adjacent vertebral members. The contouring prepares the concave-shaped endplates of the vertebral members to receive an intervertebral disc implant. Once inserted, the implants reduce or eliminate the pain and neurological deficit, and may increase the range of motion.

### SUMMARY

The present application is directed to devices and methods to shape endplates of vertebral members. In one embodiment, the device includes a guide with an elongated shaft and an enlarged distal section. An outer member may include first and second arms that are spaced a distance apart. Teeth may be positioned on outer surfaces of one or both of the first and second arms. The outer member may be movably positioned along the guide between first and second positions. The first position may include the teeth positioned over the shaft with the first and second arms being positioned a first distance apart. The second position may include the teeth positioned over the distal section with the first and second arms being positioned an expanded second distance apart. The teeth can contact the endplates of the vertebral members in the second position. The guide may include a fixed sized, or may be expandable to a variety of sizes.

### BRIEF DESCRIPTION OF THE DRAWINGS

FIG. 1A is a side schematic view illustrating a device according to one embodiment.

FIG. 1B is a side schematic view illustrating a device according to one embodiment.

FIG. 2 is an exploded side view illustrating a device according to one embodiment.

FIG. 3 is a side view illustrating a device according to one embodiment.

FIG. 4 is a cross-section view along lines IV-IV of FIG. 1B illustrating arms and a convex section according to one embodiment.

FIG. 5 is a cross-section view illustrating arms and a convex section according to one embodiment.

FIGS. 6A-6D are side schematic views of a device contouring the vertebral members according to one embodiment.

FIGS. 7A-7B are cross-section views of a device contouring the vertebral members according to one embodiment.

FIG. 8 is a cross-section view illustrating arms and a convex guide section according to one embodiment.

FIG. 9 is a cross-section view illustrating a convex guide section according to one embodiment.

### DETAILED DESCRIPTION

The present application is directed to devices for contouring endplates of vertebral members. One embodiment of the device generally includes a guide member with a convex outer surface. A second member is positioned on the exterior of the first member and includes a first surface shaped to move along the convex outer surface and a second toothed surface. The second member is moved axially along the guide member with the first surface moving along the convex outer surface of the guide member to an expanded position where the toothed section can contour the endplate of the vertebral member.

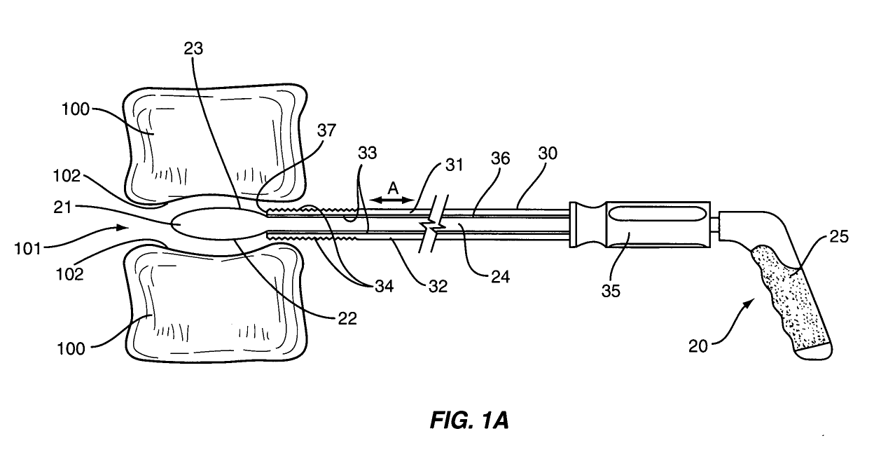

FIGS. 1A and 1B illustrate one embodiment featuring a guide 20 that is axially aligned with a second member 30. The guide 20 includes an elongated shaft 24 with a convex section 21 with first and second convex surfaces 22, 23. A handle 25 is positioned on the proximal end of the shaft 24. The second member 30 includes first and second arms 31, 32 spaced apart by a gap 36. Each of the arms 31, 32 includes an inner surface 33 the faces the guide 20. Teeth 34 are positioned on the outer surface of the arms 31, 32. A handle 25 is positioned at the proximal end of the arms 31, 32.

FIG. 1A illustrates the device in a first orientation. The convex section 21 of the guide 20 is inserted within an intervertebral space 101 formed between vertebral members 100. The second member 30 is positioned along the guide 20 with the distal end 37 positioned within the intervertebral space 101. The handles 25, 35 may remain on the exterior of the patient due to the lengths of the arms 31, 32 and shaft 24. The second member 30 is sized to axially move along the length of the guide 20. In the orientation of FIG. 1A, the second member 30 is positioned with the distal end 37 positioned inward from the convex section 21.

FIG. 1B illustrates an orientation with the second member 30 having moved distally relative to the guide 20. The distal movement causes the first and second arms 31, 32 to follow the convex surfaces 22, 23 and move radially outward. The outward movement causes the teeth 34 to contact endplates 102 of the vertebral members 100. In one embodiment, both the guide 20 and the second member 30 are reciprocated together to contour the endplates 102. In another embodiment, the second member 102 is reciprocated along the guide 20 to contour the endplates 102 of the vertebral members 100. In addition to axial movement, the second member 30 may also be rotated about the guide 20 to further contour the endplates 102. In an embodiment that uses an anterior approach to the intervertebral space 101, the axial movement is predominantly along an anterior-posterior axis and the rotational movement about the anterior-posterior axis.

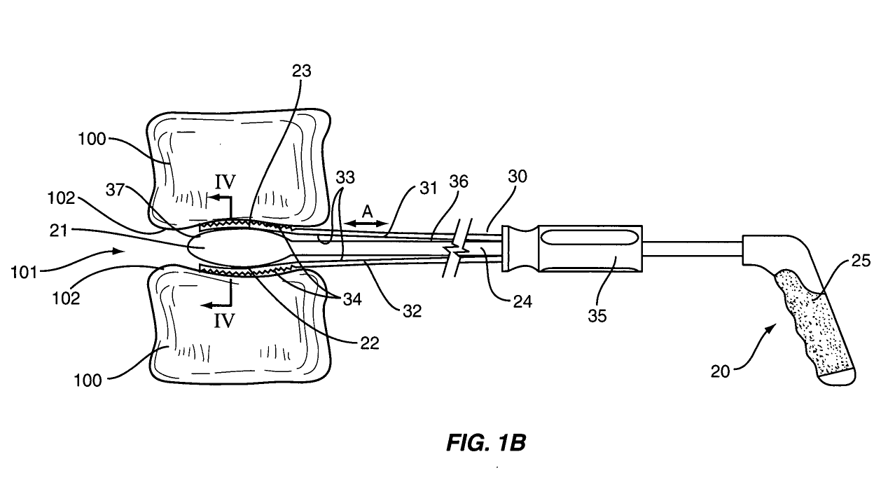

FIG. 2 illustrates an embodiment of the device in an exploded orientation. The guide 20 includes a shaft 24 that separates the convex section 21 and the handle 25. Shaft 24 may be substantially straight, or may be arcuate. Shaft 24 may include a variety of cross-sectional shapes, with one embodiment featuring a substantially circular cross-sectional shape. Shaft 24 may be constructed of a rigid material, or a flexible material that provides for bending during insertion of the convex section 21 and/or contouring movement. The handle 25 provides for grasping and manipulating the guide 20. Handle 25 may include a knurled or textured surface to prevent slipping, and may also include a pistol grip that fits the surgeon's hand. An extension 53 may be positioned along the shaft 24 and extend outward to contact the second member 30 to prevent axial movement in the distal direction. Extension 53 may include a peg that extends outward from the shaft 24 and contacts the handle 35.

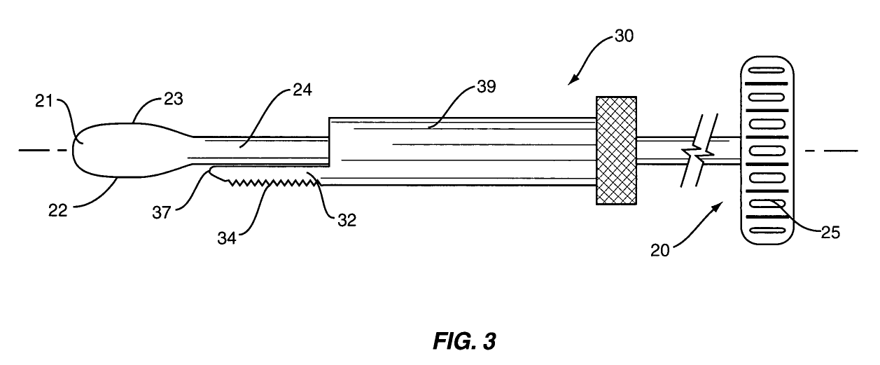

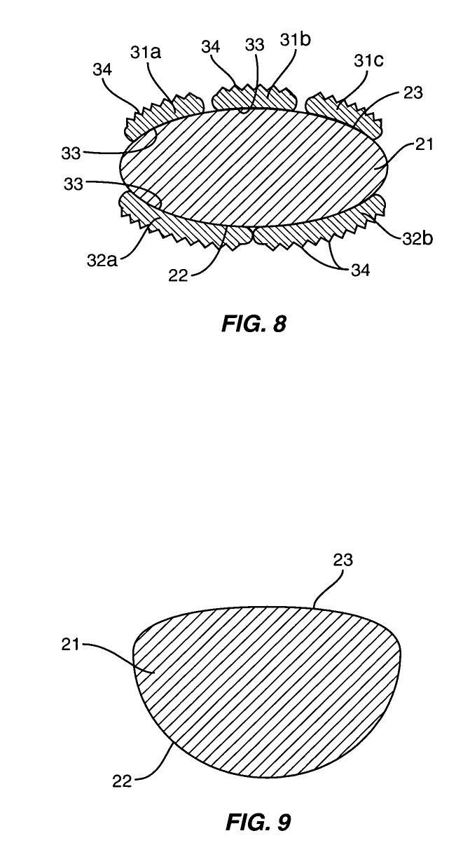

In one embodiment as illustrated in FIG. 2, the convex section 21 is positioned at the distal end of the shaft 24. The convex section 21 may also be positioned inward from the distal end. The length of the convex section 21 may vary to match the shape of the endplates 102 of the vertebral members 100 as illustrated in FIGS. 1A and 1B, although other lengths are also contemplated. Convex section 21 may be constructed from a single member as illustrated in FIG. 2, or may be constructed of two or more separate members that are in contact or spaced apart. The convex section 21 may include a convex shape in an anterior-posterior plane as illustrated in FIGS. 1A and 1B, and also in a medial-lateral plane as illustrated in FIGS. 4, 5 and 8. The surfaces 22, 23 of the convex section 21 may be substantially the same, or may be different. FIGS. 4, 5, and 8 illustrate embodiments with the convexity of the surfaces 22, 23 being substantially the same in the medial-lateral plane. FIG. 9 illustrates an embodiment with the surfaces 22, 23 including different shapes.

The second member 30 of FIG. 2 includes first and second arms 31, 32 that extend outwardly from a handle 35. The arms 31, 32 are constructed for the inner surfaces 33 to ride along the convex section 21. In one embodiment, the arms 31, 32 are constructed of a flexible material that bends during the reciprocating motion and maintains the contact. Exemplary materials may include steel, plastic, and Nitinol. In another embodiment (not illustrated), the arms 31, 32 are constructed of one or more sections that are pivotally connected together. The pivoting connections provide for the inner surfaces 33 to ride along the convex section 21. In one embodiment, arms 31, 32 are inwardly biased to contact or be in proximity to the shaft 24 in the first orientation. The biasing may be due to their construction, or a mechanical structure such as a flexible band placed around the arms 31, 32.

A gap 36 is positioned between the arms 31, 32. Gap 36 may extend from the distal end 37 proximally to the handle 35, or may extend a distance less than entirely to the handle 37. In one embodiment, gap 36 provides for the arms 31, 32 to be movable in a radial direction.

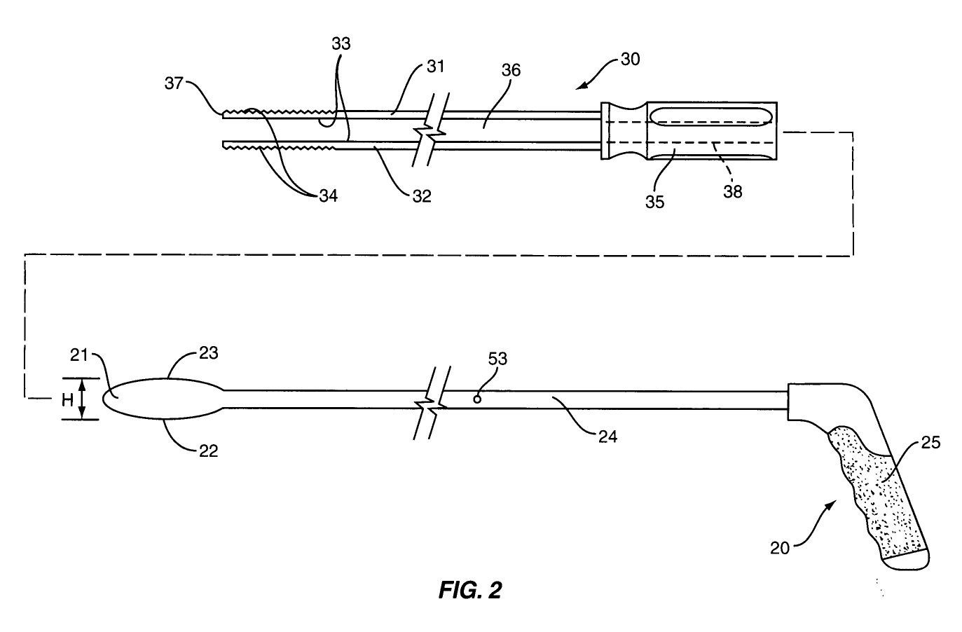

Second member 30 may also include a sleeve 39. FIG. 3 illustrates an embodiment of a sleeve 39 that extends around the shaft 24 and is spaced inwardly from the distal end 34. This embodiment further includes a single arm 34 that extends outwardly from the sleeve 39. This embodiment is designed to contour a single endplate 102 of a vertebral member 100. The first convex surface 22 guides the arm 34 to contour the vertebral member 100. The second convex surface 23 is sized to contact the opposite vertebral member 100 and maintain the position of the surface 22. The second member 30 may also be rotatable about the axis of the guide. In addition, the second member may be flipped over to contour the endplate 102 of the second vertebral member 100.

Teeth 34 are positioned on the outer surfaces of the arms 31, 32 to contour the endplates 102 of the vertebral members 100 during the movement of the second member 30. Teeth 34 may include a variety of shapes and sizes depending upon the amount of desired contour. Teeth 34 may be positioned at the distal end 37 as illustrated in FIG. 2, or may be spaced inwardly from the distal end 37 as illustrated in FIG. 3. In embodiments with two sets of teeth 34, the sets may be the same shape and size and positioned at the same distance from the distal end 37, or may be different in one or more of these attributes.

Handle 35 is sized for manipulating the second member 30. Handle 35 may include a knurled or textured outer surface to prevent slipping. A channel 38 may extend through the handle 35 and is sized to receive the shaft 24.

FIG. 4 illustrates the arms 31, 32 in contact with the convex section 21. The inner surfaces 33 of the arms 31, 32 slide along the convex surfaces 22, 23. In this embodiment, the surfaces 22, 23 are also curved in a medial-lateral plane and the arms 31, 32 flex during the contact and assume the arcuate shape. Teeth 34 extend outward from the arms 31, 32 for contouring the endplates 102 vertebral members 100. In this embodiment, teeth 34 on the first arm 31 include a different shape and configuration than teeth 34 on the second arm 32. In one embodiment as illustrated in FIGS. 1A and 1B, second member 30 is moved axially along the guide 20 in a posterior-anterior direction. Second member 30 may also be rotated about the guide 20 as illustrated by arrows X in FIG. 4. The amount of rotation may vary depending upon the shapes of the guide 20 and second member 30. In one specific embodiment, the second member 30 rotates up to about 100 in each direction.

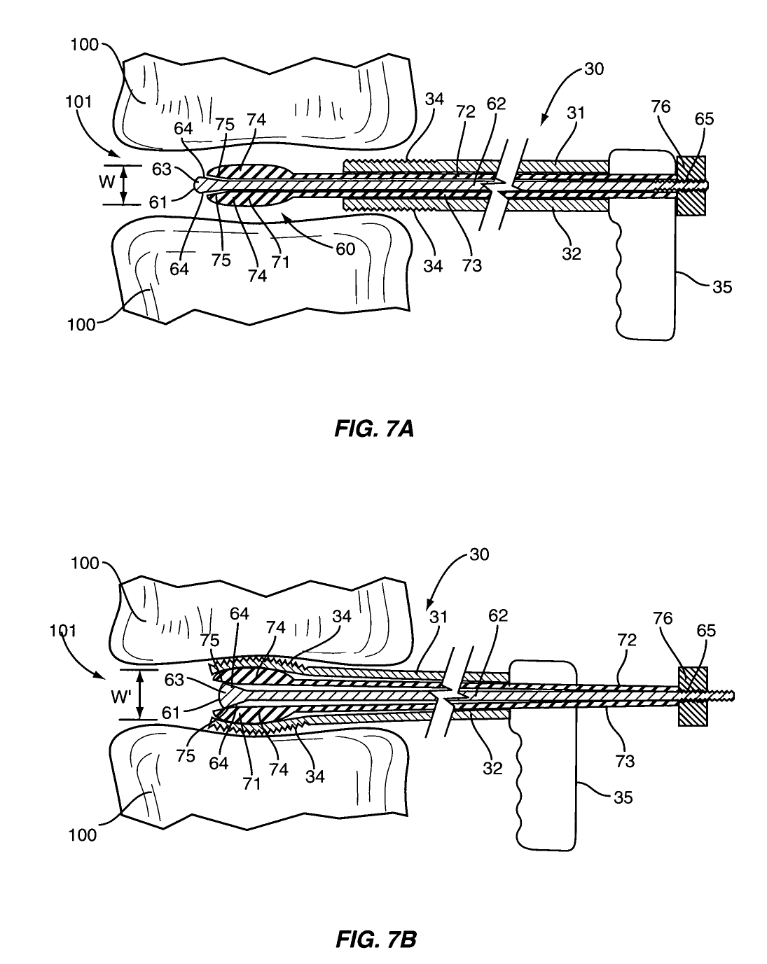

One or both arms 31, 32 may be constructed from multiple members. FIG. 8 illustrates an embodiment with the first arm 31 constructed of three separate members 31a, 31b, 31c, and second arm 32 is constructed of two members 32a, 32b. The separate members may be spaced apart, or may be in contact with adjacent members.

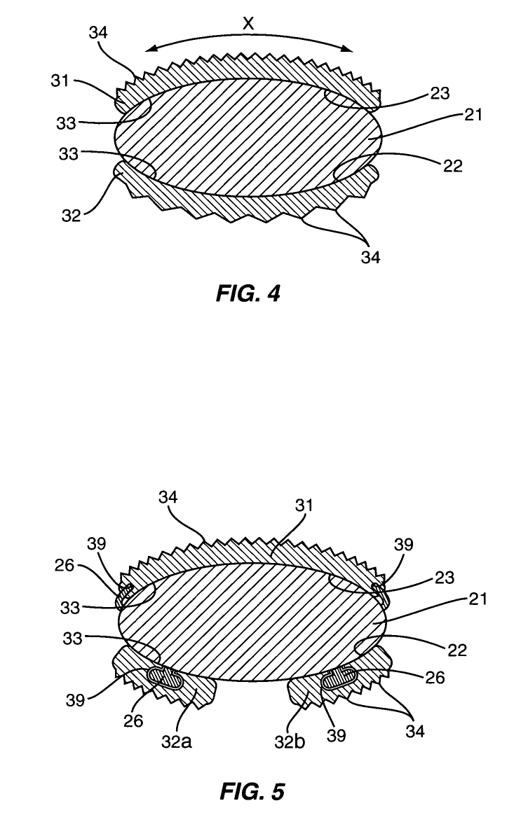

FIG. 5 illustrates an embodiment with extensions 26 extending outward from the convex section 21. The extensions 26 are shaped to engage notches 39 within the arms 31, 32 and maintain the arms 31, 32 in contact with or in proximity to the convex section 21. Extensions 26 and notches 39 may include a variety of shapes, sizes, and positional locations. The extensions 26 may be positioned on the convex section 21, or may also extend along the shaft 24. Likewise, the notches 39 may be located at the convex section 21, or also along the length of the arms 31, 32. In another embodiment (not illustrated), convex section 21 includes notches that engage with extensions on one or both arms 31, 32.

Arm 31 includes a pair of notches 39 on the lateral sides that engage with extensions 26. The arm 31 moves along the second convex surface 23 with the extensions 26 moving within the notches 39. The inferior arm 32 is divided into first and second arms 32a, 32b that move along the first convex surface 22. Each arms 32a, 32b includes a notch 39 sized and positioned to receive a corresponding extension 26.

In one embodiment, the convex section 21 is substantially rigid. The height H (see FIG. 2) is substantially constant during insertion into the intervertebral space 101. The height H is also substantially constant during the contouring movement of the second member 30. Convex section 21 may also be flexible and compressed during insertion into the intervertebral space 101. The compression causes a reduction in height H to facilitate insertion into the space 101. Once inserted, the convex section 21 may return towards the original height H.

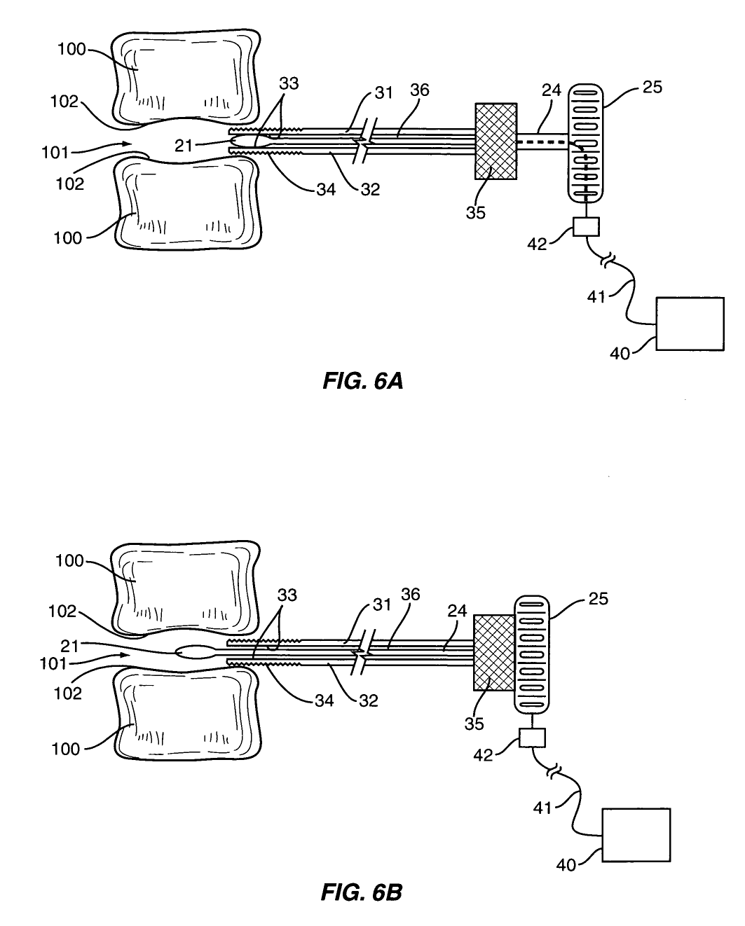

The convex section 21 may further be expandable to grow to an enlarged size once inserted within the interior space 101. FIGS. 6A-D illustrate an embodiment with an expandable convex section 21. The convex section 21 is operatively connected to a pump 40. A conduit 41 leads from the pump 40 to the handle 25, and extends through the shaft 24 to the interior of the convex section 21. The shaft 24 and handle 25 may form the conduit 41, or a separate conduit 41 may be positioned within the shaft 24 and handle 25.

FIG. 6A illustrates the convex section 21 in a reduced size with a reduced height. The reduced height provides for the first and second arms 31, 32 to be spaced in closer proximity to each other and fit within the intervertebral space 101. In one embodiment, the arms 31, 32 act as a cannula for introducing the convex section 21 into the intervertebral space 101. The convex section 21 may also be positioned within the arms 31, 32 to prevent potential damage to the section 21 during insertion.

Once the distal end 37 of the arms 31, 32 are within the intervertebral space 101, the guide 20 is moved distally and the convex section 21 is moved beyond the distal end 37. FIG. 6B illustrates this positioning caused by moving the guide 20 in a distal direction relative to the second member 30. Once inserted within the intervertebral space 101, the height of the convex section 21 is increased. Pump 40 may be activated to move a material through the conduit 41 and into the interior of the convex section 21. A variety of materials may be used for filling the convex section 21 including saline and air. A gauge 42 may be positioned along the conduit 41 or at the pump 40 to indicate the amount of material moving through the conduit 41 and the height of the convex section 21. The pump 40 may be stopped after the convex section 21 reaches the desired height as illustrated in FIG. 6C.

The second member 30 may then be axial reciprocated along the convex section 21 as illustrated in FIG. 6D. The teeth 34 contact and contour the endplates 102 of the vertebral members 100. In one embodiment, the convex section 21 is filled to a first height and a first amount of contouring is performed. Additional material is then pumped into the convex section 21 and a second amount of contouring is performed. Incremental steps may be continued until achieving the proper amount of contouring.

After completion of contouring, the material is removed from the convex section 21. This reduces the height and provides for removing the device from the intervertebral space 101. The expandable convex section 21 may be constructed of various materials including a latex inner core with a plastic or metal outer shell

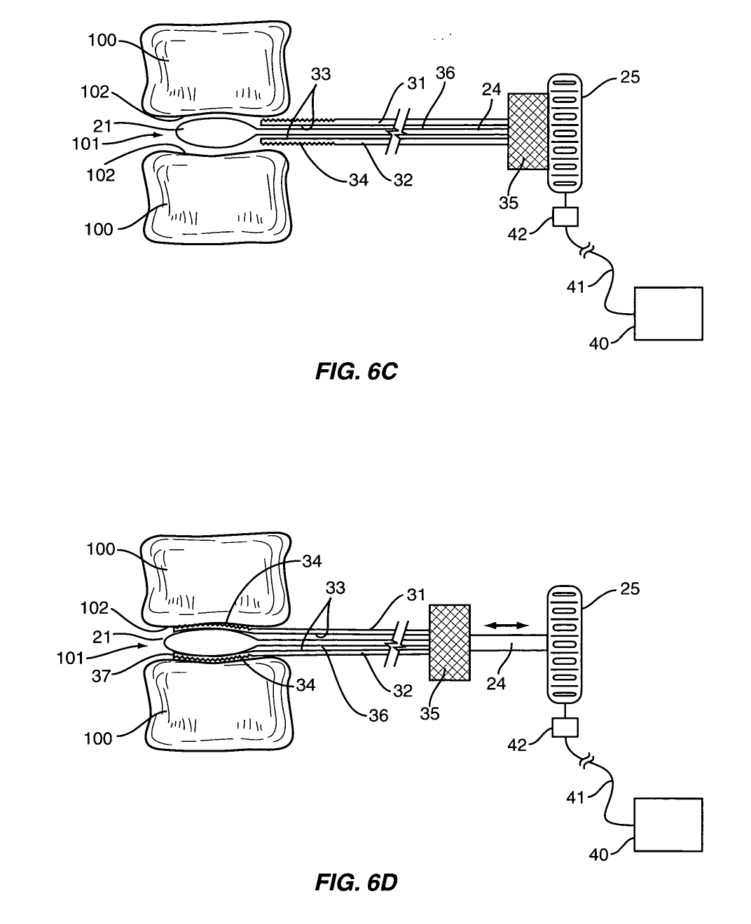

FIGS. 7A and 7B illustrate another embodiment for contouring the vertebral members 100. The device includes a guide 60 including a first member 61 and a second member 71. The first member 61 includes an elongated shaft 62 with an enlarged section 63 including first and second outwardly angled surfaces 64. Enlarged section 63 may be positioned at the distal end as illustrated in FIG. 7A, or may be spaced inward from the distal end. A proximal end of the shaft 62 includes a threaded section 65. The second member 71 includes first and second arms 72, 73 with convex sections 74. Convex sections 74 may be positioned at the distal end, or inward from the distal end. Convex sections 74 may have the same or different shapes. Each convex section 74 includes a ramped surface 75. A collar 76 is rotatably connected to the proximal end of the second member 71 and includes internal threads. Member 30 includes first and second arms 31, 32 as described above.

In a first position as illustrated in FIG. 7A, the overall height of the guide 60 is reduced to W. The enlarged section 63 extends outward from the convex sections 74 with the angled surfaces 64 of the first and second convex sections 74 being spaced from the ramped surfaces 75. In this position, the first and second convex sections 74 are positioned radially inwardly towards the shaft 62. In one embodiment, the first and second arms 72, 73 are biased radially inward either due to their construction, or a mechanical biasing mechanism such as a flexible band placed around the arms 72, 73.

In a second position as illustrated in FIG. 7B, the first member 61 has moved proximally relative to the second member 71. The angled surfaces 64 on the enlarged section 63 contact the ramped surfaces 75 of the first and second convex sections 74. The proximal movement causes the surfaces 64 to slide along the ramped surfaces 75 and expand the overall height of the guide 60 to an amount W′. The amount of expansion is dependent upon the extent of relative movement between the first and second members 61, 71. In this embodiment, the relative movement is caused by rotating the collar 76 attached to the second member 71 and engaging threads with the threaded section 65 of the first member 61. Collar 76 may also include a locking feature to prevent further relative movement between the first and second members 61, 71.

Once in the expanded position, the member 30 may be reciprocated axially and rotatably along and about the guide 60 as described above. In addition, both the guide 60 and the member 30 may be moved together to contact the teeth 34 against the vertebral members 100 thereby contouring the endplates 102. Once contouring is complete, the first member 61 is moved distally relative to the second member 71 to reduce the overall height back towards W. This reduced height facilitates removal from the intervertebral space 101.

In one embodiment, the shaft 62 is axially moved by rotating color 76 to engage the threaded section 65. Other methods of axial movement may include activation of a trigger or a lever.

One embodiment includes accessing the spine from an anterior approach. Other applications contemplate other approaches, including posterior, postero-lateral, antero-lateral and lateral approaches to the spine, and accessing other regions of the spine, including the cervical, thoracic, lumbar and/or sacral portions of the spine.

The term “distal” is generally defined as in the direction of the patient, or away from a user of a device. Conversely, “proximal” generally means away from the patient, or toward the user. Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A device to contour endplates of vertebral members comprising:
an inner member with an elongated shaft and an enlarged section;
an outer member comprising first and second arms spaced a distance apart that is greater than the shaft and less than the distal section; and
teeth positioned on outer surfaces of the first and second arms;
the outer member movably positioned along the inner member between a first position with the teeth positioned over the shaft with the first and second arms being positioned a first distance apart, and a second position with the teeth positioned over the distal section with the first and second arms being positioned an expanded second distance apart.

2. The device of claim 1, wherein the enlarged section includes first and second convex surfaces.

3. The device of claim 1, wherein the outer enlarged section is expandable between a first orientation with a first height and a second orientation with a second larger height.

4. The device of claim 1, wherein the enlarged section includes ramped first and second surfaces.

5. The device of claim 1, wherein the inner member comprises a first inner member with an enlarged distal end and a second inner member including convex outer surfaces and angled inner surfaces, the first inner member being axially movable relative to the second inner member to adjust an overall height of the inner member.

6. The device of claim 1, wherein the outer member is rotatably movable along the inner member.

7. The device of claim 1, wherein the first and second arms are flexible and flex outward when the outer member moves from the first position to the second position.

8. The device of claim 1, wherein the enlarged section of the inner member is expandable and further comprising a conduit that extends through the shaft to move material into the enlarged section.

9. The device of claim 1, wherein the enlarged section is positioned at a distal end of the inner member.

10. A device to contour endplates of vertebral members comprising:
an inner member with a proximal section and an enlarged distal section;
an outer member comprising an inner surface that faces towards the inner member and an outer surface including teeth that face towards one of the endplates;
the outer member movably positioned along the inner member between a first position with the inner member at a proximal position relative to the outer member with the teeth positioned away from the distal section and positioned a first distance from a centerline of the proximal section, and a second position with the inner member at a distal position relative to the outer member with the teeth positioned over the distal section and positioned a second greater distance from the centerline of the proximal section.

11. The device of claim 10, wherein the inner surface contacts the distal section of the inner member when moving from the first position to the second position.

12. A device to contour endplates of vertebral members comprising:
a guide including an enlarged section with first and second angled surfaces;
a member movably engaged with the guide and including a first arm with a first inner surface that moves along the first angled surface and a second arm with a second inner surface that moves along the second angled surface; and
each of the first and second arms including teeth on an outer surface that contour the vertebral members during movement of the member along the enlarged section.

13. A device to contour endplates of vertebral members comprising:
a guide including first and second convex surfaces;
a member movably engaged with the guide and including a first arm with a first inner surface that moves along the first convex surface and a second arm with a second inner surface that moves along the second convex surface; and
each of the first and second arms including teeth on an outer surface that contour the vertebral members during movement of the member along the first and second convex surfaces.

14. The device of claim 13, wherein the first and second convex surfaces include a common length and shape.

15. The device of claim 13, wherein the guide includes an extension that extends outward and mates with a notch within one of the first and second arms.

16. The device of claim 13, wherein the guide is rigid.

17. The device of claim 13, wherein the first and second arms are flexible with the inner surfaces remaining in contact with the first and second convex surfaces during movement of the member along the guide.

18. The device of claim 13, wherein one of the first and second arms is divided into first and second sections.

19. The device of claim 13, wherein the first and second convex surfaces are positioned on a guide section of the guide, the guide section is expandable between a first orientation with a first reduced height and a second orientation with a second height.

20. The device of claim 19, further comprising a pump to move material into the guide section to expand the guide section from the first orientation to the second orientation.

21. A device to contour endplates of vertebral members comprising:
a guide including a first convex surface positioned at a distal end of a shaft;
an elongated member positioned on an exterior of the guide and including an inner surface that moves along the first convex surface; and
teeth positioned on an outer surface of the arm to contour one of the vertebral members during movement of the elongated member along the guide.

22. The device of claim 21, further comprising a sleeve positioned at a proximal end of the arm that extends around the shaft.

23. The device of claim 21, wherein the guide includes a second surface that is substantially convex.

24. A method of contouring an intervertebral space between vertebral members, the method comprising the steps of:
inserting a distal section of a guide within a central section of the intervertebral space;
moving a member axially along a first section of the guide;
contacting an inner surface of the member against an enlarged distal section of the guide and moving the member outward away from the central section and into contact with one of the vertebral members; and
reciprocating the member within the intervertebral space and contouring the vertebral member with teeth positioned on an outer surface of the member.

25. The method of claim 24, wherein the step of reciprocating the member within the intervertebral space and contouring the vertebral member with the teeth positioned on the outer surface of the member comprises maintaining the position of the guide and reciprocating the member along the guide.

26. The method of claim 24, wherein the step of moving the member axially along a first section of the guide comprises contacting the inner surface of the member against the first section of the guide.

27. The method of claim 24, wherein the step of reciprocating the member within the intervertebral space and contouring the vertebral member with the teeth positioned on the outer surface of the member comprises reciprocating the member and the guide within the intervertebral space.

28. The method of claim 24, further comprising expanding a width of the distal section of the guide after inserting the distal end within the central section of the intervertebral space.
